(19) United States
(12) Patent Application Publication
(10) Pub. No.: US 2021/0169773 A1
(43) Pub. Date: Jun. 10, 2021

(54) Skin Enhancing Composition

(71) Applicants: Brian Nourian, Beverly Hills, CA (US); Sharona Rafaeloff, Beverly Hills, CA (US); Parvaneh Rafaeloff, Beverly Hills, CA (US)

(72) Inventors: Brian Nourian, Beverly Hills, CA (US); Sharona Rafaeloff, Beverly Hills, CA (US); Parvaneh Rafaeloff, Beverly Hills, CA (US)

(21) Appl. No.: 16/708,055

(22) Filed: Dec. 9, 2019

(51) Int. Cl. A61K 8/9789; A61K 8/64; A61K 8/73; A61K 8/49; A61K 8/34; A61K 8/362; A61Q 19/00

(52) U.S. Cl. CPC A61K 8/9789; A61K 8/64; A61K 8/735; A61Q 19/00; A61K 8/347; A61K 8/4973; A61K 8/362; A61K 8/498

(57) ABSTRACT

A skin enhancing composition is provided. The skin enhancing composition comprises filtered water, at least two antioxidants, one or more plant components, an anti-inflammatory agent, and an alpha hydroxy acid. The antioxidants neutralize endogenous free radicals, thereby reducing cell damage and the visual effects of aging. The antioxidants comprise cannabinoid distillates, l-glutathione, d-alpha tocopherol, trans-resveratrol, or citric acid. The plant components are derived from any whole or partial fruit, vegetable or other plant, or an extract thereof. The anti-inflammatory agent, such as ascorbic acid, supports white blood cells and improves the health and appearance of skin by evening out skin tone, shielding the skin from visible impacts of pollution, and improving hydration. The alpha hydroxy acids improve skin evenness and tone and reduce wrinkles and other signs of aging. In some embodiments, additional vitamins, minerals, one or more natural flavors, and one or more natural colors are also provided.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to skincare compositions and, more particularly, to a skin enhancing composition comprising ingredients which promote collagen production, increase skin hydration, improve skin smoothness and tone, and prevent damage caused by free radicals, in addition to other integumentary, cardiovascular, musculoskeletal, gastrointestinal and neurological benefits.

### BACKGROUND

The human body, and specifically, the skin and immune system physiologically decline with aging. External sources, such as ultraviolet light exposure or pollution, can advance these effects by causing inflammation and other harmful insults. Signs of aging commonly manifest as fine lines, wrinkles, age spots, loss of volume, melasma, dullness and loss of elasticity. Such signs are undesirable to individuals who desire to appear as youthful as possible.

Numerous over-the-counter anti-aging products are commercially available, all of which boast properties purportedly tied to skin enhancement. These cosmeceuticals not only beautify the skin but also improve its functioning by maintaining keratin structure, encouraging collagen growth, and combatting the harmful effects of free radicals. For example, many compositions contain anti-inflammatory additives such as vitamins A, C and E. As another example, supplemental collagen has become popularized over the last few years.

Some inventors have patented or attempted to patent various solutions to humans' aging woes. For instance, Chinese Patent No. 103874431 for a glutathione-containing health drink comprises hyaluronic acid, glutathione, collagen and various vitamins and other compounds. This teaching is deficient because there remains doubt as to the efficacy and safety of collagen, whether ingestible or delivered as a topical solution. Another proposed solution, United States Patent Publication No. 2010/0004330 discloses a mixture of antioxidants which provide anti-aging, anti-cancer, anti-heart disease, and vision-enhancing benefits. This publication fails to contain other beneficial antioxidants and other skin enhancing ingredients, such as sodium hyaluronate, as taught by Applicant.

There remains a need for a skin enhancing beverage which has anti-oxidative and anti-inflammatory properties and that not only beautifies but also nourishes the skin.

### SUMMARY

The present disclosure is directed to skin enhancing compositions that cause human skin to appear visibly clearer, tighter, smoother and brighter. More particularly, the composition may provide anti-oxidative as well as anti-inflammatory properties. In this manner, the composition may delay the onset of aging, reduce the effects of aging, and improve the appearance of skin.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the skin enhancing composition may comprise filtered water, one or more antioxidants, one or more plant components, sodium hyaluronate, one or more anti-inflammatory agents, and an alpha hydroxy acid. In addition, in some embodiments, the composition may further comprise one or more vitamins or minerals, one or more natural flavors, and one or more natural colors.

The one or more antioxidants may comprise alternately or in combination with one another, one or more cannabinoid distillates, l-glutathione, d-alpha tocopherol, trans-resveratrol, and citric acid. The antioxidants may reduce oxidative stress within the body by neutralizing free radicals, thereby reducing or mitigating disease and inflammation.

In embodiments where the composition may comprise one or more cannabinoid distillates, the cannabinoid distillates may be water-soluble, odorless, flavorless, and color-free. The cannabinoid distillates may further comprise sorbitol, modified food starch, coconut oil, cannabidiol (CBD), and tetrahydrocannabinol (THC). In certain embodiments, the cannabinoid distillates may comprise a 1:20 ratio of CBD to THC. In other embodiments, the CBD and the THC may be present in different ratios.

In embodiments comprising 1-glutathione, the l-glutathione may detoxify xenobiotics and heavy metals. The l-glutathione may also reduce oxidative and consequently, disease. The l-glutathione may thereby increase skin elasticity by reducing wrinkles and lightening the skin.

In certain embodiments, the at least two antioxidants may comprise d-alpha tocopherol. The d-alpha tocopherol may neutralize endogenous free radicals effectively in that it is a fat-soluble antioxidant. In other embodiments, another stable form of vitamin E may be substituted or used in combination with the d-alpha tocopherol. For instance, other forms of alpha-tocopherol, gamma-tocopherol, or mixed tocopherols may be used.

Trans-resveratrol (otherwise known as 3, 4^{I}, 5-tri-hydroxystilbene) may also be provided as an antioxidant. In addition, trans-resveratrol may provide cognitive, bone, gut health, cardiovascular, anti-carcinogenic, and anti-aging benefits. Alternatively, other stilbenes may be substituted or used in combination with trans-resveratrol.

Finally, citric acid may also be provided as an antioxidant, as well as, an alkalizing and flavoring agent. Citric acid may neutralize the pH of the skin, thereby evening out rough or uneven patches of skin. In further embodiments, citric acid may act as a preserving agent as well.

The one or more plant components may comprise a whole or a partial fruit, vegetable, or other plant. By way of example and not limitation, the one or more plant components may comprise red dragon fruit, mango, cucumber, or watermelon. In some embodiments, the plant may be an edible plant. In some of such embodiments, the edible plant may be a flowering plant, such as a rose, lavender, or turmeric.

Also, in some embodiments, the one or more plant components may be cold-pressed, thermal-pressed, pureed, blended, or otherwise physical formed into a liquid form. In alternate embodiments, the one or more plant components may be formed into a solid form, such as a powder. In still other embodiments, the one or more plant components may be chemically formed into a liquid form. In some of these embodiments, the one or more plant components may comprise extractions of vitamins, minerals or other nutrients from the fruit, vegetable, or other plant.

In some embodiments of the present invention, hyaluronic acid may be provided. In some of these embodiments, hyaluronic acid may be combined with a salt, such as sodium, to form sodium hyaluronate. Sodium hyaluronate may bind water and carry essential nutrients various parts of the human body. In this manner, sodium hyaluronate may aid in skin health, in addition to joint and muscle care.

The one or more anti-inflammatory agents may comprise ascorbic acid. Ascorbic acid may aid the inflammatory system by helping white blood cells to function better. Ascorbic acid also aids the health and appearance of skin by evening out skin tone, shielding the skin from visible impacts of pollution, and improving hydration. Moreover, ascorbic acid may aid in the growth, development, and repair of all bodily tissues.

The alpha hydroxy acid may comprise one or more of malic acid, glycolic acid, lactic acid, tartaric acid, citric acid, or mandelic acid. Alpha hydroxy acids may reduce signs of aging and wrinkles by increasing collagen production. Additionally, alpha hydroxy acids may exfoliate the skin by removing dead skin cells and improve skin smoothness and tone.

Lastly, the skin enhancing composition may comprise additional vitamins, minerals, natural flavors, or natural colors. By way of example, and not limitation, the composition may comprise vitamin C, vitamin E, or vitamin H. Further, the composition may comprise natural flavors, which enhance flavor but do not contribute nutritional value. The one or more natural flavors may be extracted from plant or animal sources.

One or more of the above-disclosed embodiments, in addition to certain alternatives, are provided in further detail below with reference to the attached figures. The disclosed subject matter is not, however, limited to any particular embodiment disclosed.

### OBJECTS AND ADVANTAGES

Several advantages of one or more aspects are to provide a skin enhancing composition that:
(a) Neutralizes endogenous free radicals, thereby reducing oxidative stress and disease-causing cellular damage;
(b) Possesses anti-inflammatory properties;
(c) Increases skin elasticity;
(d) Reduces the appearance of wrinkles and fine lines;
(e) Promotes healthy turnover and renewal of keratinocytes;
(f) Supports collagen production;
(g) Evens out skin tone and texture;
(h) Shields skin from visible impacts of pollution;
(i) Improves skin hydration;
(j) Aids in the treatment of acne; and
(k) Treats a variety of cardiovascular, cognitive, ocular, digestive, and other tissue conditions.

These and other advantages of one or more aspects will become apparent from consideration of the ensuing description. Although the description above contains many specifics, these should not be construed as limiting the scope of the embodiments but as merely providing illustrations of some of several embodiments. Thus, the scope of the embodiments should be determined by the claims that are appended and their legal equivalents, rather than by the examples given. The description of the invention which follows should not be construed as limiting the invention to the examples shown and described, because those skilled in the art to which this invention pertains will be able to devise other forms thereof within the ambit of the appended claims.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

A skin enhancing composition is provided that, in some embodiments, increases skin elasticity, reduces the appearance of wrinkles and fine lines, promotes collagen production and healthy keratinocyte turnover, and evens out skin tone and texture. The composition may comprise filtered water, one or more antioxidants, one or more plant components, sodium hyaluronate, one or more anti-inflammatory agents, and an alpha hydroxy acid. Further, in certain embodiments, the composition may also comprise one or more natural flavors, one or more natural colors, and one or more vitamins or minerals.

Primarily, filtered water may comprise the majority of the percent weight of the skin enhancing composition. In other embodiments, filtered water may be provided to a lesser percent weight. One of ordinary skill in the art will understand that filtered water may help transport other dissolved ingredients, such as those discussed in more detail below, across the cellular membrane, and therefore into the cells. Thus, filtered water may act as a cellular carrier for various compounds.

In some embodiments, the one or more antioxidants may neutralize free radicals, which are endogenous, unstable molecules that lead to cell damage and disease. By neutralizing the free radicals, the one or more antioxidants may thereby prevent cellular damage and inflammation. In turn, the antioxidants may prevent heart disease, cancer, diabetes, and arthritis. Moreover, the antioxidants may protect white blood cells, thereby boosting the immune system. The one or more antioxidants may comprise one or more cannabinoid distillates, l-glutathione, d-alpha tocopherol, trans-resveratrol, and citric acid. A person of ordinary skill in the art will recognize that any of the aforementioned antioxidants may be provided separately or in combination with one another.

The one or more cannabinoid distillates may be water-soluble, odorless, flavorless, and color-less. The cannabinoid distillates may be quickly absorbed into the bloodstream and thereby provide fast-action. In addition, the cannabinoid distillates may provide anti-inflammatory benefits and also aid in chronic pain. Further, the cannabinoid distillates may be non-psychoactive and may also mitigate insomnia and some epilepsy syndromes. In some embodiments, the cannabinoid distillates may further comprise sorbitol, modified food starch, coconut oil, cannabidiol (CBD), and tetrahydrocannabinol (THC). In such embodiments, cannabinoid distillates may comprise a 1:20 ratio of CBD to THC. In other embodiments, the ratio of CBD to THC may be greater. Meanwhile, in alternate embodiments, the ratio of CBD to THC may be lesser.

L-glutathione may also be provided as an antioxidant. L-glutathione may comprise three amino acids: cysteine, glutamic acid, and glycine. L-glutathione may increase skin elasticity, reduce wrinkles, and cause skin lightening. Additionally, the l-glutathione may detoxify xenobiotics and heavy metals, reduce oxidative stress (and therefore, disease), and neutralize hydrogen peroxide. Moreover, l-glutathione may maintain the integrity of blood cells, aid in carbohydrate metabolism, and regulate insulin and blood glucose levels.

The composition may further comprise d-alpha tocopherol or another stable form of vitamin E. D-alpha tocopherol may be a fat-soluble antioxidant and neutralize endogenous free radicals. In this manner, d-alpha tocopherol may have anti-aging effects and also treat skin conditions like acne, psoriasis, and ultraviolet skin damage. D-alpha tocopherol may also treat cardiovascular disease, ocular conditions, diabetes, and cancer. In alternate embodiments, another form of vitamin E may be included, such as other forms of alpha tocopherol, gamma-tocopherol, beta-tocopherol, or delta-tocopherol.

As another antioxidant, trans-resveratrol may be provided. Trans-resveratrol may be cold-water dispersible, which enhances bioavailability. Trans-resveratrol may delay age-related diseases, promote healthy aging, and therefore, may provide anti-aging effects. Trans-resveratrol may also provide neuroprotective, bone, digestive, cardiovascular, and anti-carcinogenic benefits. In other embodiments, other stilbenes may be substituted for or used in combination with trans-resveratrol.

In some embodiments, citric acid may also be provided. Citric acid may be an antioxidant, alkalizing agent, and flavoring agent. Citric acid may neutralize the skin's pH and, in turn, may even out rough, uneven portions of skin. Citric acid may further be provided as a preserving agent.

One or more plant components may also form a portion of the skin enhancing composition. One of ordinary skill in the art will understand the one or more plant components may comprise a whole or a partial fruit, vegetable, other plant, or even, an extract thereof. For instance, in embodiments where the one or more plant components comprise a fruit or a vegetable, the one or more plant components may be a red dragon fruit, a white dragon fruit, mango, cucumber, or watermelon. Virtually any other fruits or vegetables may be provided as the one or more plant components. The one or more plant components may also be a flowering plant, such as a rose, lavender, or turmeric.

In embodiments wherein the one or more plant components comprise red dragon fruit, the red dragon fruit may provide a number of benefits. Red dragon fruit may be low in calories and contain zero cholesterol. Red dragon fruit may provide anti-aging benefits. Red dragon fruit may also act as an antioxidant, neutralizing free radicals which lead to cell damage. Moreover, red dragon fruit may naturally comprise other essential vitamins and minerals, such as iron, magnesium, vitamin C, vitamin E, potassium, and calcium.

In embodiments wherein the one or more plant components may comprise roses, the roses may add significant hydration and moisture to the skin. Roses, therefore, may treat dry, irritated, red, itchy skin and also maintain the moisture barrier. Roses may also act as an antioxidant by helping to strengthen skin cells and regenerate skin tissues. Roses may further smooth the appearance of wrinkles, reduce the appearance of age spots, and aid with collagen production. In certain embodiments, the one or more plant components may comprise red dragon fruit and roses.

In alternate embodiments, as mentioned above, the one or more plant components may comprise mango. Mango may serve to hydrate the skin and improve skin tone. Mango may further provide antioxidative properties by defending the skin against damage from free radicals. In some embodiments, mango may also boost the immune system, promote collagen production, repair DNA, and prevent acne breakouts.

In other embodiments, the one or more plant components may comprise turmeric, which may provide glow and luster to the skin. Turmeric may also decrease inflammation and oxidation, causing wounds and scars to heal more quickly. Turmeric may also aid in other skin conditions, including psoriasis, eczema, alopecia, lichen planus, as well as others. In further embodiments, the one or more plant components comprise mango and turmeric.

In embodiments wherein the one or more plant components may comprise watermelon, watermelon may hydrate the skin and act as a skin toner. Indeed, watermelon may naturally shrink bodily tissues, thereby refreshing the skin. Watermelon may also prevent the skin from aging by reducing free radicals and prevent oil overproduction from sebaceous glands.

As a final example, the one or more plant components may comprise cucumber. Cucumber may provide vitamin C and caffeic acid to the skin, which may, in turn, reduce inflamed or irrated skin. Additionally, cucumber may comprise a high water content and therefore may hydrate the skin. Cucumber may also contain silica, which may firm up the skin. In certain embodiments, the one or more plant components may comprise watermelon and cucumber.

Sodium hyaluronate, or hyaluronic acid, may also be provided in the composition. In such embodiments, sodium hyaluronate may comprise natural hyaluronic acid in sodium salt. Sodium hyaluronate may promote healthy turnover and renewal of keratinocytes. Additionally, sodium hyaluronate may support healthy collagen, hydrate the skin, and moderate the appearance of fine lines and age spots. Sodium hyaluronate may also assist in retaining the synovial fluid of the joints, which provide shock absorption and lubrication for movement, thereby aiding in joint and muscle care.

In certain embodiments, the one or more anti-inflammatory agents may comprise ascorbic acid. Ascorbic acid may help white blood cells to function better. Ascorbic acid also aids the health and appearance of skin by evening out skin tone, shielding the skin from visible impacts of pollution, and improving hydration. Moreover, ascorbic acid may aid in the growth, development, and repair of all bodily tissues. Ascorbic acid may further aid in the formation of collagen, absorption of iron, and maintenance of cartilage.

In some embodiments, the alpha hydroxy acid may comprise malic acid. In other embodiments, the alpha hydroxy acid may comprise one or more of glycolic acid, lactic acid, tartaric acid, citric acid, mandelic acid, 2-hydroxyoctanoic acid, and 2-hydroxydecanoic acid. Alpha hydroxy acids may reduce signs of aging and wrinkles by increasing collagen production. In addition, alpha hydroxy acids may exfoliate the skin by removing dead skin cells and improve skin smoothness and tone. Alpha hydroxy acids may also aid in the treatment of acne, improve skin hydration, and facilitate energy production.

The skin enhancing composition may comprise additional ingredients as well. In some embodiments, the skin enhancing composition may comprise additional vitamins and/or minerals. For instance, the composition may comprise vitamin C, vitamin E, or vitamin H. As another example, the composition may comprise caffeine or green tea. In certain embodiments, the composition may comprise one or more natural flavors and/or one or more natural colors. The natural flavors may enhance flavor and may be created from substances extracted from plant or animal sources, including spices, fruit or fruit juice, vegetable or vegetable juice, edible yeast, herbs, bark, buds, root leaves, plant material, dairy products, eggs, meat, poultry, or seafood.

A person of ordinary skill in the art will recognize that the skin enhancing composition may be provided in a liquid or a solid form. In embodiments wherein the composition may be provided in liquid form, the composition may be delivered orally as a beverage. In alternate embodiments wherein the composition is a liquid, the composition may be delivered intravenously through the bloodstream. In embodiments wherein the composition may be provided in solid form, the composition may be dissolvable in liquid as a powder, or may be delivered orally in pill or tablet form.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### Examples of the Preferred Embodiment

In order to more fully teach what the Applicant regards as his invention, the following example is given. It should be understood that the formulations set forth in this Example is not to be construed as limiting the scope of the invention, except so far as they yield a skin enhancing composition having the desired properties and characteristics. The following ingredients are an example of Applicant's invention with the percentages being given by weight of the composition:

| Ingredient | Percentage |
|---|---|
| Filtered water | 93.7 |
| Plant components | 5.0 |
| Cannabinoid distillates | 0.02 |
| L-glutathione | 0.03 |
| Sodium hyaluronate | 0.03 |
| D-alpha tocopherol | 0.006 |
| Trans-resveratrol | 0.03 |
| Ascorbic acid | 0.02 |
| Malic acid | 0.02 |
| Citric acid | 0.03 |
| Natural flavors | 1.1 |

### CONCLUSIONS, RAMIFICATIONS, AND SCOPE

While certain embodiments of the invention have been illustrated and described, various modifications are contemplated and can be made without departing from the spirit and scope of the invention. For example, other plant components may be utilized, other than those described above. Accordingly, it is intended that the invention not be limited, except as by the appended claims.

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the skin enhancing composition with which that terminology is associated. In general, the terms used in the following claims should not be constructed to limit the skin enhancing composition to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the skin enhancing composition is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the skin enhancing composition.

What is claimed is:

1. A liquid beauty composition, comprising
filtered water;
one or more cannabinoid distillates;
l-glutathione;
one or more plant components;
sodium hyaluronate;
d-alpha tocopherol;
trans-resveratrol;
ascorbic acid;
malic acid;
citric acid; and
one or more natural flavors.

2. The liquid beauty composition of claim 1, wherein the one or more cannabinoid distillates comprises: sorbitrol, modified food starch, coconut oil, cannabidiol and tetrahydrocannabinol.

3. The liquid beauty composition of claim 2, further comprising:
up to 10.0 mg cannabidiol; and
up to 0.5 mg tetrahydrocannabinol.

4. The liquid beauty composition of claim 1, wherein the one or more plant components comprises a fruit, a vegetable or an edible plant.

5. The liquid beauty composition of claim 4, wherein the fruit is a red dragon fruit, a watermelon, or a mango.

6. The liquid beauty composition of claim 4, wherein the vegetable is a cucumber.

7. The liquid beauty composition of claim 4, wherein the edible plant is a rose.

8. The liquid beauty composition of claim 1, further comprising:
up to 94.6% wt. filtered water;
up to 0.03% wt. one or more cannabinoid distillates;
up to 0.04% wt. l-glutathione;
up to 5.0% wt. one or more plant components;
up to 0.03% wt. sodium hyaluronate;
up to 0.007% wt. d-alpha tocopherol;
up to 0.04% wt. trans-resveratrol;
up to 0.03% wt. ascorbic acid;
up to 0.04% wt. malic acid;
up to 0.06% wt. citric acid; and
up to 0.12% wt. one or more natural flavors.

9. The liquid beauty composition of claim 1, further comprising one or more natural colors.

10. The liquid beauty composition of claim 9, wherein the one or more natural colors are defined by the one or more plant components' color.

11. A skin enhancing composition, comprising:
filtered water;
one or more cannabinoid distillates;
l-glutathione;
one or more plant components;
sodium hyaluronate;
d-alpha tocopherol;
trans-resveratrol;
one or more alpha hydroxy acids; and
one or more vitamins.

12. The composition of claim 11, wherein the one or more plant components are selected from red dragon fruit, rose, watermelon, cucumber, mango, and turmeric.

13. The composition of claim 11, wherein the one or more alpha hydroxy acids are selected from malic acid, citric acid, glycolic acid, and lactic acid.

14. The composition of claim 11, wherein the one or more vitamins are vitamin C or vitamin E.

15. The composition of claim 11, further comprising one or more natural flavors and one or more natural colors.

16. The composition of claim 15, further comprising:
between 92.9 and 94.5% wt. filtered water;
between 0.025 and 0.027% wt. one or more cannabinoid distillates;
between 0.034 and 0.037% wt. l-glutathione;
between 4.0 and 5.0% wt. one or more plant components;
between 0.02 and 0.03% wt. sodium hyaluronate;
between 0.004 and 0.006% wt. d-alpha tocopherol;
between 0.028 and 0.03% wt. trans-resveratrol;
between 0.02 and 0.022% wt. ascorbic acid;
between 0.02 and 0.05% wt. malic acid;
between 0.02 and 0.06% wt. citric acid; and
between 0.15 and 0.24% wt. one or more natural flavors and one or more natural colors.

17. A liquid skin enhancing composition, comprising:
filtered water;
at least two antioxidants;
at least two plant components;
sodium hyaluronate;
at least one anti-inflammatory agent; and
an alpha hydroxy acid.

18. The liquid skin enhancing composition of claim 17, wherein the at least two antioxidants are cannabinoid distillates, l-glutathione, d-alpha tocopherol, trans-resveratrol, or citric acid.

19. The liquid skin enhancing composition of claim 17, wherein the at least one anti-inflammatory agent is ascorbic acid.

20. The liquid skin enhancing composition of claim 17, wherein the alpha hydroxy acid is malic acid.
